(19) United States
(12) Patent Application Publication
(10) Pub. No.: US 2019/0038473 A1
(43) Pub. Date: Feb. 7, 2019

(54) Dressing Augmentation To Promote Epithelialization

(71) Applicant: KCI Licensing, Inc., San Antonio, TX (US)

(72) Inventors: James A. LUCKEMEYER, San Antonio, TX (US); James COURAGE, San Antonio, TX (US); Timothy Mark ROBINSON, Shillingstone (GB); Christopher Brian LOCKE, Bournemouth (GB)

(21) Appl. No.: 16/073,863

(22) PCT Filed: Mar. 6, 2017
(86) PCT No.: PCT/US2017/020875

§ 371 Date: Jul. 30, 2018

Related U.S. Application Data
(60) Provisional application No. 62/310,443, filed on Mar. 18, 2016.

(51) Int. Cl. A61F 13/02; A61M 1/00; A61F 13/00; A61L 15/26; A61L 15/40; A61L 15/44; A61L 15/32; A61F 15/00; A61N 1/10

(52) U.S. Cl. CPC A61F 13/0216; A61L 2300/414; A61F 13/0259; A61F 13/0269; A61F 13/00063; A61F 13/00059; A61L 15/26; A61L 15/40; A61L 15/44; A61L 15/32; A61F 15/002; A61F 13/0283; A61N 1/10; A61F 2013/00174; A61F 2013/00182; A61L 2300/252; A61M 1/0088

(57) ABSTRACT

A treatment system for applying negative-pressure therapy to a wound site is disclosed. The treatment system may include a non-adherent material for applying to the perimeter of the wound site. In some embodiments, the treatment system may include a tape, a tissue interface, a sealing member, and a negative-pressure subsystem. The tape material may provide a smooth surface along the perimeter of the wound site for protecting the wound edges from stresses and for promoting epithelialization.

### RELATED APPLICATION

This present invention claims the benefit, under 35 USC 119(e), of the filing of U.S. Provisional Patent Application Ser. No. 62/310,443, entitled “Dressing Augmentation to Promote Epithelialization,” filed Mar. 18, 2016, which is incorporated herein by reference for all purposes.

### TECHNICAL FIELD

The invention set forth in the appended claims relates generally to tissue treatment systems and more particularly, but without limitation, to apparatuses and methods for promoting epithelialization at the perimeter of wounds during negative-pressure therapy.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

While the clinical benefits of negative-pressure therapy are widely known, improvements to negative-pressure systems, components, and processes may benefit healthcare providers and patients.

### BRIEF SUMMARY

New and useful systems, apparatuses, and methods for providing fluid seals around wound sites in a negative-pressure therapy environment are set forth in the appended claims. Illustrative embodiments are also provided to enable a person skilled in the art to make and use the claimed subject matter.

In some embodiments, a strip may be applied to the perimeter of a wound site as a tape. The strip may provide a smooth surface along the perimeter of a wound site for supporting epithelialization earlier in the wound healing process.

More generally, in some embodiments, a system for treating a tissue site may include a tape, a tissue interface, a sealing member, and a negative-pressure subsystem. The tape may include a flexible material adapted to be placed in contact with a perimeter of the tissue site, and may include a non-adherent upper surface and a tissue-facing surface. The tissue interface may include a granulating surface, and the sealing member may be transparent and having an adherent material for coupling to an epidermis and operable to form a fluid seal. The sealing member may be adapted to not substantially adhere to the non-adherent upper surface of the tape. In some example embodiments, the tape may further include a reinforcement material placed in contact with the flexible material and adapted to provide structural support to the tape. In some example embodiments, the flexible material of the tape may include a hydrogel, and the reinforcement material may be positioned on the surface of the flexile material or be substantially contained within the flexible material. In some embodiments, the tape may also include a separating layer, which may be a silicone layer, applied to a surface of the flexible material.

Alternatively or additionally, other example embodiments may include a tape including a flexible material adapted to be placed in contact with the perimeter of a wound and a reinforcement material placed in contact with the flexible material and adapted to provide structural support to the flexible material. In some example embodiments, the flexible material may include or consist essentially of a hydrogel, and in some embodiments may be a porous, cationic hydrogel. In some embodiments, the flexible material may additionally or alternatively include additional components, such as human dermis cells, electrically-conductive material, temperature- or pH-sensitive elements, or other materials.

In some embodiments, a tape dispenser may include a tape spool, a tape adapted to be contained in a roll by the tape spool, and a handle operatively fixed to the tape spool. The tape may include a flexible material for disposing adjacent to a wound edge and a reinforcement material for providing structural support to the tape. The handle may be configured for allowing a user to apply the tape around a perimeter of a wound. In some embodiments, the tape may further include a separating layer having a silicone material and adapted to allow the tape to be formed into the roll.

A method of manufacturing a tape is also described herein, wherein some example embodiments may include forming a flexible material for disposing adjacent to a wound edge, forming a reinforcement material for providing structural support to the tape, and disposing the reinforcement material on a first surface of the flexible material. In some embodiments, the method of manufacturing may further include forming a separating layer adapted to reduce attachment of an adhesive drape to the tape and disposing the separating layer on a second surface of the flexible material.

In still other embodiments, a method of treating a wound site may include disposing a tape adjacent to a wound edge, forming a fluid seal over the wound site and the tape, and fluidly coupling a negative-pressure source to the wound site. The tape may include a flexible material for disposing adjacent to a wound edge and a reinforcement material for providing structural support to the tape. The method may further include disposing a manifold adjacent to the wound site prior to forming a fluid seal over the wound site and the tape.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a functional block diagram of an example embodiment of a therapy system that can deliver negative pressure to a tissue site in accordance with this specification;

FIG. 2 is an assembly view illustrating additional details that may be associated with some example embodiments of a wound dressing in the therapy system of FIG. 1;

FIG. 3 is a schematic diagram of an example embodiment of a device that can be used to apply a tape to a tissue site; and

FIG. 4 is a schematic diagram of an alternative example embodiment of a device that can be used to apply a tape to a tissue site.

### DESCRIPTION OF EXAMPLE EMBODIMENTS

The following description of example embodiments provides information that enables a person skilled in the art to make and use the subject matter set forth in the appended claims, but may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

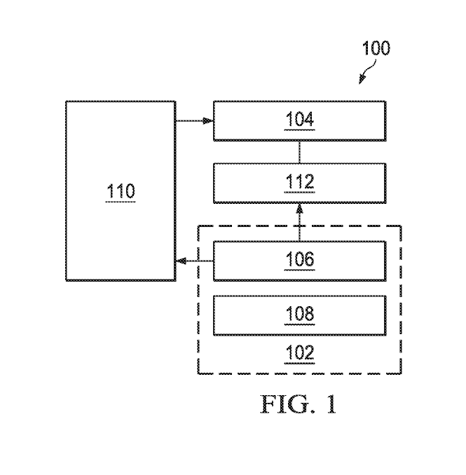

FIG. 1 is a simplified functional block diagram of an example embodiment of a therapy system 100 that can provide negative-pressure therapy in accordance with this specification.

The term “tissue site” in this context broadly refers to a wound, defect, or other treatment target located on or within tissue, including but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted.

The therapy system 100 may include a negative-pressure supply, which may include or be configured to be coupled to a distribution component, such as a dressing. In general, a distribution component may refer to any complementary or ancillary component configured to be fluidly coupled to a negative-pressure supply in a fluid path between a negative-pressure supply and a tissue site. A distribution component is preferably detachable, and may be disposable, reusable, or recyclable. For example, a dressing 102 may be fluidly coupled to a negative-pressure source 104, as illustrated in FIG. 1. The dressing 102 may include a cover 106 and a tissue interface 108. A regulator or a controller, such as a controller 110, may also be coupled to the negative-pressure source 104.

In some embodiments, a dressing interface may facilitate coupling the negative-pressure source 104 to the dressing 102. For example, such a dressing interface may be a T.R.A.C.® Pad or Sensa T.R.A.C.® Pad available from Kinetic Concepts, Inc. of San Antonio, Tex. The therapy system 100 may optionally include a fluid container, such as a container 112, coupled to the dressing 102 and to the negative-pressure source 104.

Components may be fluidly coupled to each other to provide a path for transferring fluids (i.e., liquid and/or gas) between the components. For example, components may be fluidly coupled through a fluid conductor, such as a tube. A “tube,” as used herein, broadly includes a tube, pipe, hose, conduit, or other structure with one or more lumina adapted to convey a fluid between two ends. Typically, a tube is an elongated, cylindrical structure with some flexibility, but the geometry and rigidity may vary. In some embodiments, components may also be coupled by virtue of physical proximity, being integral to a single structure, or being formed from the same piece of material. Moreover, some fluid conductors may be molded into or otherwise integrally combined with other components. Coupling may also include mechanical, thermal, electrical, or chemical coupling (such as a chemical bond) in some contexts. For example, a tube may mechanically and fluidly couple the dressing 102 to the container 112 in some embodiments.

In general, components of the therapy system 100 may be coupled directly or indirectly. For example, the negative-pressure source 104 may be directly coupled to the controller 110, and may be indirectly coupled to the dressing 102 through the container 112.

The fluid mechanics of using a negative-pressure source to reduce pressure in another component or location, such as within a sealed therapeutic environment, can be mathematically complex. However, the basic principles of fluid mechanics applicable to negative-pressure therapy are generally well-known to those skilled in the art, and the process of reducing pressure may be described illustratively herein as “delivering,” “distributing,” or “generating” negative pressure, for example.

In general, exudates and other fluids flow toward lower pressure along a fluid path. Thus, the term “downstream” typically implies something in a fluid path relatively closer to a source of negative pressure or further away from a source of positive pressure. Conversely, the term “upstream” implies something relatively further away from a source of negative pressure or closer to a source of positive pressure. Similarly, it may be convenient to describe certain features in terms of fluid “inlet” or “outlet” in such a frame of reference. This orientation is generally presumed for purposes of describing various features and components herein. However, the fluid path may also be reversed in some applications (such as by substituting a positive-pressure source for a negative-pressure source) and this descriptive convention should not be construed as a limiting convention.

“Negative pressure” generally refers to a pressure less than a local ambient pressure, such as the ambient pressure in a local environment external to a sealed therapeutic environment provided by the dressing 102. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. Similarly, references to increases in negative pressure typically refer to a decrease in absolute pressure, while decreases in negative pressure typically refer to an increase in absolute pressure. While the amount and nature of negative pressure applied to a tissue site may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −75 mm Hg (−9.9 kPa) and −300 mm Hg (−39.9 kPa).

A negative-pressure supply, such as the negative-pressure source 104, may be a reservoir of air at a negative pressure, or may be a manual or electrically-powered device that can reduce the pressure in a sealed volume, such as a vacuum pump, a suction pump, a wall suction port available at many healthcare facilities, or a micro-pump, for example. A negative-pressure supply may be housed within or used in conjunction with other components, such as sensors, processing units, alarm indicators, memory, databases, software, display devices, or user interfaces that further facilitate therapy. For example, in some embodiments, the negative-pressure source 104 may be combined with the controller 110 and other components into a therapy unit. A negative-pressure supply may also have one or more supply ports configured to facilitate coupling and de-coupling the negative-pressure supply to one or more distribution components.

The tissue interface 108 can be generally adapted to contact a tissue site. The tissue interface 108 may be partially or fully in contact with the tissue site. If the tissue site is a wound, for example, the tissue interface 108 may partially or completely fill the wound, or may be placed over the wound. The tissue interface 108 may take many forms, and may have many sizes, shapes, or thicknesses depending on a variety of factors, such as the type of treatment being implemented or the nature and size of a tissue site. For example, the size and shape of the tissue interface 108 may be adapted to the contours of deep and irregular shaped tissue sites. Moreover, any or all of the surfaces of the tissue interface 108 may have projections or an uneven, course, or jagged profile that can induce strains and stresses on a tissue site, which can promote granulation at the tissue site.

In some embodiments, the tissue interface 108 may be a manifold. A “manifold” in this context generally includes any substance or structure providing a plurality of pathways adapted to collect or distribute fluid across a tissue site under pressure. For example, a manifold may be adapted to receive negative pressure from a source and distribute negative pressure through multiple apertures across a tissue site, which may have the effect of collecting fluid from across a tissue site and drawing the fluid toward the source. In some embodiments, the fluid path may be reversed or a secondary fluid path may be provided to facilitate delivering fluid across a tissue site.

In some illustrative embodiments, the pathways of a manifold may be interconnected to improve distribution or collection of fluids across a tissue site. In some illustrative embodiments, a manifold may be a porous foam material having interconnected cells or pores. For example, cellular foam, open-cell foam, reticulated foam, porous tissue collections, and other porous material such as gauze or felted mat generally include pores, edges, and/or walls adapted to form interconnected fluid channels. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

The average pore size of a foam may vary according to needs of a prescribed therapy. For example, in some embodiments, the tissue interface 108 may be a foam having pore sizes in a range of 400-600 microns. The tensile strength of the tissue interface 108 may also vary according to needs of a prescribed therapy. For example, the tensile strength of a foam may be increased for instillation of topical treatment solutions. In one non-limiting example, the tissue interface 108 may be an open-cell, reticulated polyurethane foam such as GranuFoam® dressing or VeraFlo® foam, both available from Kinetic Concepts, Inc. of San Antonio, Tex.

The tissue interface 108 may be either hydrophobic or hydrophilic. In an example in which the tissue interface 108 may be hydrophilic, the tissue interface 108 may also wick fluid away from a tissue site, while continuing to distribute negative pressure to the tissue site. The wicking properties of the tissue interface 108 may draw fluid away from a tissue site by capillary flow or other wicking mechanisms. An example of a hydrophilic foam is a polyvinyl alcohol, open-cell foam such as V.A.C. WhiteFoam® dressing available from Kinetic Concepts, Inc. of San Antonio, Tex. Other hydrophilic foams may include those made from polyether. Other foams that may exhibit hydrophilic characteristics include hydrophobic foams that have been treated or coated to provide hydrophilicity.

The tissue interface 108 may further promote granulation at a tissue site when pressure within the sealed therapeutic environment is reduced. For example, the tissue interface 108 may have at least one granulating surface that can induce microstrains and stresses at a tissue site if negative pressure is applied through the tissue interface 108. A granulating surface generally provides an uneven, coarse, or jagged profile.

In some embodiments, the tissue interface 108 may be constructed from bioresorbable materials. Suitable bioresorbable materials may include, without limitation, a polymeric blend of polylactic acid (PLA) and polyglycolic acid (PGA). The polymeric blend may also include without limitation polycarbonates, polyfumarates, and capralactones. The tissue interface 108 may further serve as a scaffold for new cell-growth, or a scaffold material may be used in conjunction with the tissue interface 108 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

In some embodiments, the cover 106 may provide a bacterial barrier and protection from physical trauma. The cover 106 may also be constructed from a material that can reduce evaporative losses and provide a fluid seal between two components or two environments, such as between a therapeutic environment and a local external environment. The cover 106 may be, for example, an elastomeric film or membrane that can provide a seal adequate to maintain a negative pressure at a tissue site for a given negative-pressure source. The cover 106 may have a high moisture-vapor transmission rate (MVTR) in some applications. For example, the MVTR may be at least 300 g/m̂2 per twenty-four hours in some embodiments. In some example embodiments, the cover 106 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained.

An attachment device may be used to attach the cover 106 to an attachment surface, such as undamaged epidermis, a gasket, or another cover. The attachment device may take many forms. For example, an attachment device may be a medically-acceptable, pressure-sensitive adhesive that extends about a periphery, a portion, or an entire sealing member. In some embodiments, for example, some or all of the cover 106 may be coated with an acrylic adhesive having a coating weight between 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

A controller, such as the controller 110, may be a microprocessor or computer programmed to operate one or more components of the therapy system 100, such as the negative-pressure source 104. In some embodiments, for example, the controller 110 may be a microcontroller, which generally comprises an integrated circuit containing a processor core and a memory programmed to directly or indirectly control one or more operating parameters of the therapy system 100. Operating parameters may include the power applied to the negative-pressure source 104, the pressure generated by the negative-pressure source 104, or the pressure distributed to the tissue interface 108, for example. The controller 110 is also preferably configured to receive one or more input signals, such as a feedback signal, and programmed to modify one or more operating parameters based on the input signals. For example, the controller 110 may receive signals from one or more sensors, such as a pressure sensor or electric sensor, that are operable to detect or measure one or more operating parameters of the therapy system 100.

The container 112 is representative of a container, canister, pouch, or other storage component, which can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with negative-pressure therapy.

In operation, the tissue interface 108 may be placed within, over, on, or otherwise proximate to a tissue site. The cover 106 may be placed over the tissue interface 108 and sealed to an attachment surface near the tissue site. For example, the cover 106 may be sealed to undamaged epidermis peripheral to a tissue site. Thus, the dressing 102 can provide a sealed therapeutic environment proximate to a tissue site, substantially isolated from the external environment, and the negative-pressure source 104 can reduce the pressure in the sealed therapeutic environment. Negative pressure applied across the tissue site through the tissue interface 108 in the sealed therapeutic environment can induce macrostrain and microstrain in the tissue site, as well as remove exudates and other fluids from the tissue site, which can be collected in container 112.

Wound therapy systems, such as the therapy system 100, can be used to promote granulation of a tissue site, and treatment protocols often involve following negative-pressure therapy with other dressings to complete epithelial closure of the tissue site. During negative-pressure therapy, which may be applied with negative-pressure systems, such as therapy system 100, epithelialization may actually be delayed due to several factors. First, caregivers often have a tendency to over pack wounds with foam, which can lead to irritation of the wound edges. Second, wound edges may experience drape shear due to vacuum forces, in addition to patient movement. Furthermore, damage to epithelial growth often occurs during drape and foam removal, such as during routine wound dressing changes. With deep wounds, the primary pain sensations may be around the wound edges. Thus, there is a need for a wound dressing enhancement that protects the edges of a wound site from disruption, and thereby accelerates the naturally-occurring epithelialization and healing processes of a wound site, while also minimizing irritation and discomfort for patients. As disclosed herein, the therapy system 100 may address these needs and others. For example, the therapy system 100 may provide a smooth, sheer-less surface along the perimeter of a wound, without compromising granulation in the middle. By actively supporting epithelialization earlier in the wound healing process, the overall time to closure of the wound should be reduced.

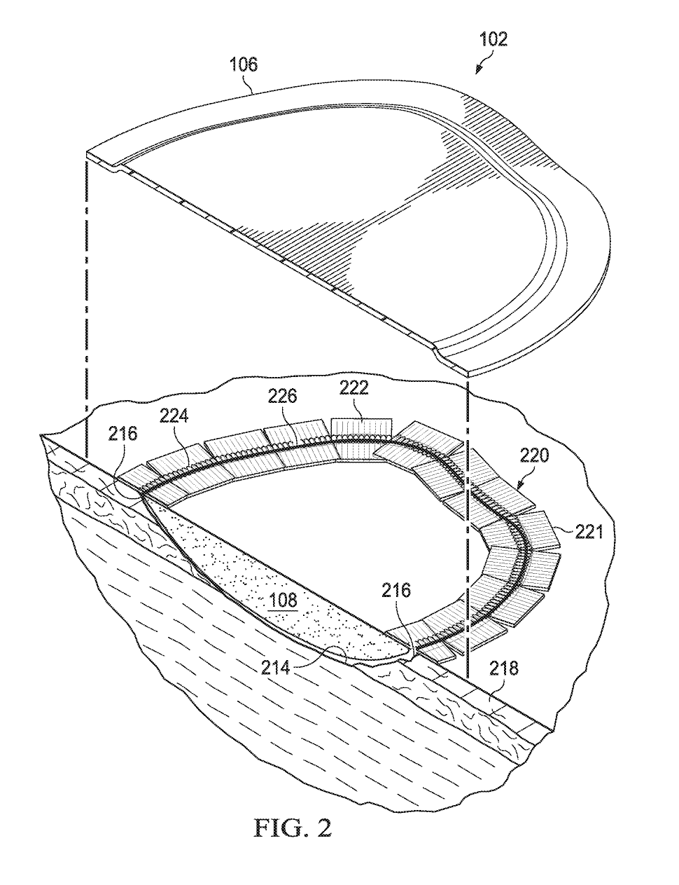

FIG. 2 is a schematic view illustrating additional details that may be associated with some example embodiments of the dressing 102. In the example embodiment of FIG. 2, the dressing 102 generally includes the cover 106, the tissue interface 108, as well as an epithelialization interface in the form of a tape 220. The tape 220 may be applied around the circumference of a wound site 214, along the wound edge 216. Some embodiments of the tape 220 may comprise or be substantially formed from a biocompatible material, which may have a smooth, low-friction surface. For example, the tape 220 may be formed from a hydrogel having a low viscosity, which may allow for any shear forces applied to the tape 220 to be dispersed and/or absorbed by the tape 220, and largely not transmitted to the wound site 214.

Additionally, the tape 220 may offer semi-adherent or substantially non-adherent surfaces, which may allow the tape 220 to remain in place while the other components of the dressing 102, such as the cover 106, are changed. For example, the tape 220 may include a tissue-facing surface and an upper surface. The upper surface of the tape 220 may be semi-adherent in order to support a high seal with the cover 106, however the bond between the upper surface of the tape 220 and the cover 106 may be insufficient to cause the tape 220 to be removed with the cover 106 and/or the tissue interface 108, when these components of the dressing 102 are changed. The tape 220 may also be capable of maintaining a moist environment. For example, in some embodiments, the tape 220 may be adapted to absorb excess moisture around the edges of the wound site 214, as well as donate water back to the wound site 214 in more dry conditions. Therefore, in some preferred embodiments, the tape 220 may include a material that can absorb at least its own weight in water.

In the example embodiment of FIG. 2, the tape 220 may be in the form of tape strips, such as segments 221, and may be formed from a hydrogel 222 and reinforced with an optional internal textile scrim 224. In some embodiments, the hydrogel 222 may be a hydrophilic material with a viscosity range of 15 shore 000 to 40 shore 000. The hydrogel 222 may have a low-friction surface and may be adapted to maintain a moist environment at a wound site, such as wound site 214, and thus may not hinder the migration of cells. In some embodiments, the hydrogel 222 may include materials such as the LUDLOW product, available from Covidien, or the AQUACLEAR product, available from HARTMANN USA, Inc. In other embodiments, the tape 220 may be formed from alternatives to a hydrogel, and may include one or more of the following materials: cyanoacrylate sealant, fibrin sealant, silicone sealant, collagen sealant, hydrocolloid sealant, albumin sealant, hot-melt-type surgical paste, polymer gels such as polyurethanes, polyolefins, acrylics, and cellulosic (carboxymethyl cellulose), and alginates. The materials typically have a smooth, low-friction surface, are capable of maintaining a moist environment, and are biocompatible. Example embodiments of the tape 220 may include Adaptic® product from Systagenix Wound Management, ALGIDERM product, available from C. R. Bard, Inc., and/or AQUACEL product, available from ConvaTec, Inc.

The textile scrim 224 may prevent excessive stretching of the tape 220 and may provide support for the edge of the tape 220, as it can overhang the wound edge 216 into the wound site 214 in some applications. The textile scrim 224 may take various forms, but in some preferred embodiments may be a mesh or net, and may include fringes that span throughout the tape 220. In some embodiments, the textile scrim 224 may be an elastic woven or non-woven material. In some embodiments, the textile scrim 224 may comprise or consist essentially of one or more polyurethane elastomers, for example, elastane or spandex materials. The textile scrim may be of various dimensions and weights, and in some embodiments, may be about 25 g/m². A tape centerline marker 226 may also be incorporated into the tape 220, which may assist a user with following the perimeter of a wound when applying the tape 220 to the wound edge 216. In some embodiments, the tape centerline marker 226 may be a colored thread.

The tape 220 may be of any suitable width or thickness, but in some preferred embodiments, may be in the range of 20 mm to 35 mm wide, with a thickness of 1 mm to 2 mm. In some embodiments, the width dimension may be influenced by usability studies of the optimal offset of the tape 220 along the inside edge of a wound, such as wound site 214, which may encourage decreased sizing of a tissue interface (to avoid over-packing the wound bed), such as tissue interface 108, as wound healing progresses. In other embodiments, the tape 220 may include the feature of a dimensional bias to the tape centerline marker 226 away from a center line of the tape 220. Such a feature may help guide and train users. For example, users may be instructed to put the wide edge of the tape 220 towards the inside of the wound edge 216 for large wounds, or the narrow edge of the tape 220 to the inside of the wound edge 216 for small wounds. Further, in some embodiments, the tape 220 may be offered in different color choices, which may allow users to personalize the product, as well as may be used to color-code different sizes or varieties of the tape 220.

The tape 220 may provide various structural benefits to the perimeter of a wound, such as along the wound edge 216. For example, the tape 220 may prevent tension from the cover 106 or other component of the dressing 102 from being transferred to the wound edge 216, thereby preventing shear forces from contacting the wound edge 216. Furthermore, in some embodiments, the tape 220 may be formed with adequate strength to resist collapse under a compressive force transmitted by the cover 106 when negative pressure is applied to the dressing 102.

Additionally, the physical structure and properties of the tape 220 may offer significant benefits for application to non-linear wounds, such as circular or irregularly-shaped wounds. Whereas typically, attempting to apply a linear-type tape or material around bends or corners would result in an uneven surface with ridges, possibly leading to discontinuous contact with a tissue site, and consequently, leaks, the properties of the tape 220 may allow it to conform to a variety of differently-shaped wounds. For example, as depicted in FIG. 2, the tape 220 may be in the form of a segmented structure, which may improve the ability of the tape 220 to flex, bend, or otherwise conform to the perimeter of a wound. In some embodiments, the tape 220 may include portions, such as segments 221, that are independently positionable, for closely tracking an irregularly-shaped border of a wound. In some instances, the tape 220 may include perforations between the segments 221, which may allow the segments 221 to be individually shaped to the contours of a wound. Additionally, in some embodiments, the segments 221 may be individually removable. To provide even greater flexibility, the tape 220, may be offered with segments 221 having a variety of sizes. For example, in some embodiments, the tape 220 may have segments 221 that are of a shorter length, therefore allowing for even a more precise fit around edges of small and/or particularly irregularly-shaped wounds. In some additional embodiments of the tape 220, some segments 221 may be of a greater length, while some may be of a smaller length, and may be in an alternating arrangement.

Another benefit of the segmented structure of the tape 220 is that, as compared to a more traditional configuration of adhesive tape being applied to the external surface of a wound drape for securing the drape to a patient, the tape 220 may be much more user-friendly. For example, while portions of a traditional adhesive tape may have a high tendency to stick to each other, and consequently leading to the tape being ruined and discarded, the segments 221 of the tape 220 can be easily positioned around, as well as in contact with, each other without becoming permanently stuck to each other. As a result, the tape 220 may be much more forgiving to a user who may be inexperienced or who may need to make adjustments during the application of the tape 220, in order to achieve the best application around a given wound site.

Furthermore, in some embodiments, the textile scrim 224 of the tape 220 may be at least partially elastic or stretchable in nature, which may further assist a user, such as a clinician, with tracing irregular borders of a wound, such as the wound edge 216. Thus, at least in some embodiments, due to the combination of potentially elastic properties and the small width of the textile scrim 224 relative to the overall tape 220, the tape 220 may be particularly effective at making a close contact around sharp corners of a wound site, despite originating as a linear material. As a result, an improved seal may be formed between the tape 220 and the cover 106, when applied, which may help reduce or prevent fluid leaks.

Still referring to FIG. 2, the tissue interface 108 may be placed in contact with the wound site 214, inside of the boundary formed by the tape 220 that is applied to the wound edge 216. The cover 106 may be positioned over the tape 220 and tissue interface 108, and a portion of the epidermis 218. As discussed in further detail above, the cover 106 may be attached to the epidermis 218 surrounding the wound site 214, using a variety of conventional attachment mechanisms.

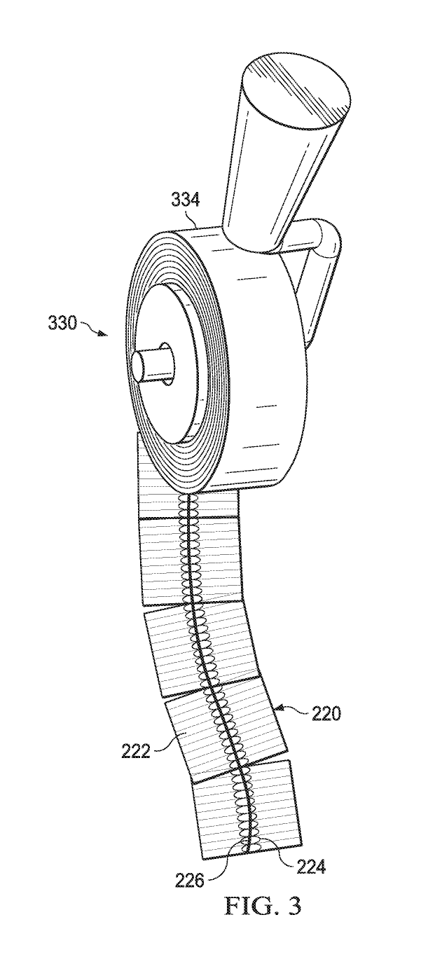

FIG. 3 is a perspective view of an example embodiment of an applicator that may be used with the tape 220. The applicator may offer various benefits, for example, such as allowing the tape 220 to be applied more quickly. In the example of FIG. 3, the applicator is a tape roller 330. In operation, the tape 220 may be applied around a perimeter of a wound by rolling the tape roller 330 around a wound edge. The tape centerline marker 226 may assist with tracking the wound edge 216. The applied tape 220 may then provide a visual cue for proper sizing of a tissue interface, such as the tissue interface 108, to fit inside of the tape edge, which can discourage over-packing the wound site 214 and avoid contact of the tissue interface 108 with the wound edge 216. In some embodiments the tape roller 330 may include components that are injection-molded polymers, such as PC (polycarbonate), ABS (acrylonitrile-butadiene-styrene), or PC-ABS blends. PC-ABS blends may include the C1200HF, C2800, or C2950 products, as supplied by SABIC, or alternatively, may include the Emerge 7570 or Pulse 2000EZ products, as supplied by TRINSEO. In other embodiments, the tape roller 330 may include a construction including combinations of rigid plastics and thermoplastic elastomers. The applicator may be a mini tape roller, such as the tape roller 330, and may be a disposable medical product, which may be furnished sterile. Further, in some embodiments, the entire applicator assembly, along with any remaining tape, may be disposed after a single patient use. In some circumstances, remaining tape may be used to assist with sealing drape leaks during subsequent drape overlay. To aid such use at a later time, the spool, such as tape spool 334, could be designed to easily pop-off from the tape roller, such as tape roller 330.

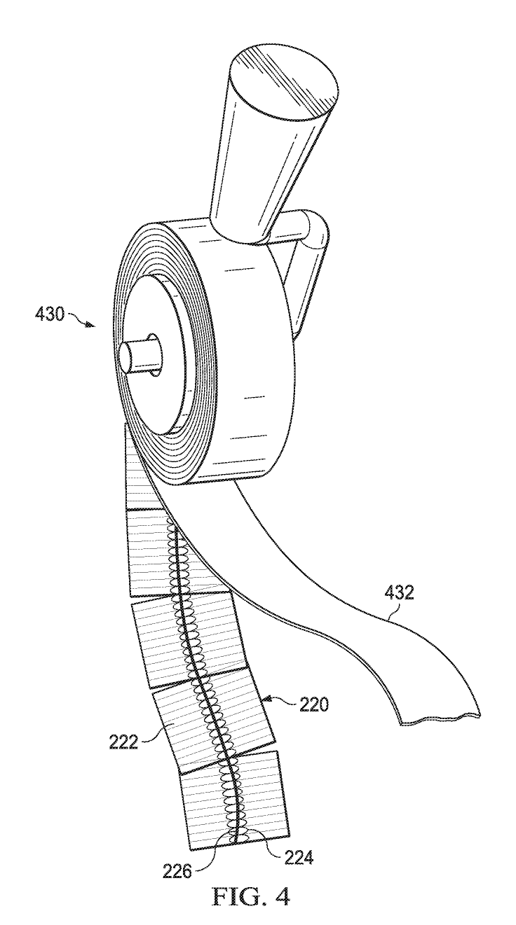

FIG. 4 is a perspective view of an alternative embodiment of a tape roller 430 that may be used to apply tape, such as tape 220, to a tissue site. The tape roller 430 may be used with the tape 220, as well as an additional separating layer 432 for ease of applying a single layer of tape 220 at a time from the tape roller 430. For example, in some embodiments, the separating layer 432 may be a thin silicone layer. When applied to a wound edge, the separating layer 432 may also provide a silicone surface for reducing attachment of the cover 106. Thus, the tape 220 may remain in place during a dressing change, which may further reduce trauma to new epithelial cell growth. In some embodiments, the upper sealing face of the separating layer 432 may be made from a silicone gel, elastomer, or other material unlikely to bond well to an acrylic adhesive. In some embodiments, the separating layer 432 may have an upper layer of self-lubricating silicone, which may excrete small volumes of liquid silicone over the wear duration. Such materials are known in the art and may be obtained from suppliers such as WACKER, MOMENTIVE, and DOW. In some embodiments, the upper sealing face of the separating layer 432 may include scrim material or support fabric, which may allow the separating layer 432 to mechanically link with adhesive on the surface of a cover, while also allowing easy separation of the separating layer 432 from the cover, such as cover 106.

The separating layer 432 may also provide a seal for the dressing 102, as there may be some degree of bond between the cover 106 and the separating layer 432. However, this bond may be insufficient to cause the separating layer 432, or the tape 220 as a whole, to be removed with the cover 106 when portions of the dressing are changed. In some embodiments, the separating layer 432 may be left in-place for several dressing changes. For example, the tape 220 may be changed every second or third dressing change. The tape 220 may also have the inherent feature of providing guidance during periodic reapplications if the tape 220 is reapplied with a smaller perimeter around a wound site, thus enforcing the resizing of a wound interface, such as a foam, to shrink as the wound healing progresses.

In addition to the tape embodiments already discussed, the tape concept can be extended to include various additional features and therapies. For example, in some embodiments, human dermis tissue may also be included as part of the tape, as hydrogels have been shown in the art to be a suitable media for allowing epithelial cell growth and extracellular matrix formation. In such embodiments, tape strips may be preferred to a roll of tape, as the geometry of the tape strips may be more suitable for inclusion of human dermal cells. Dermal-spiked tape strips may be placed around a wound perimeter, or selectively in areas in or around a wound site that are ready for epithelialization. In some embodiments, additional tape strips, or tape applied from a tape roller, may be used to trace areas ready for epithelialization, particularly in cases of larger overall wound sites.

In other alternative embodiments, the tape may incorporate features to allow electrical stimulation only in the tape area. In such embodiments, the electrical stimulation may be provided by galvanic cells or an externally-applied source. For example, in some embodiments, a conductive element, such as a carbon-impregnated fiber, conductive polymer thread, or thin metallic wire may be placed near each edge of the tape such that an electrical current source can be applied to provide electrical stimulation across the tape, in the desired direction of epithelialization along the wound edge. In other embodiments, the conductor materials could be chosen to create a galvanic cell across the tape width, taking advantage of the salts and ions in wound fluid. In some embodiments, for electrical stimulation, the electrical source could be an external battery for continuous stimulation, or in other alternative embodiments, the electrical source could be a battery with an electronic circuit to generate a preferred waveform and control timing for repeated stimulation sessions. In such embodiments, the electronic circuit may be a small electronic circuit that may be placed at the wound site. Still in other alternative embodiments, electrical contacts may be included for attachment of an electronic-stimulation therapy device for periodic treatment sessions performed by a caregiver.

In some alternative embodiments, the tape construct may also incorporate a silk mat designed to slowly release a therapeutic substance, such as growth factors, for example, epidermal growth factor (EGF). In other alternative embodiments, nanostructures may similarly be incorporated into the tape construct, which may adapted to release drugs and other active agents. In still other alternative embodiments, antimicrobial or anti-biofilm features could be added to the tape construct. For example, there are hydrogels known in the art designed to break down biofilms by damaging microbe membranes through electrostatic interaction of positively-charged gel and negatively-charged bacterial or fungal cells. In other embodiments, electro-spun fiber structures designed to attract and capture infectious bacteria may be incorporated in the tape construct.

Further, in some alternative embodiments, the tape may employ a color-changing feature to alert caregivers of an infection present at the wound site. For example, the tape may include a temperature-sensitive material that may change colors in response to temperature. In other embodiments, the tape may include electro-spun fiber structures designed to attract and capture infectious bacteria, which may change color with pH change. Such features may be particularly helpful for untrained users or caregivers in the home healthcare market. In some additional embodiments, the hydrogel for tape materials could also be designed to provide mechanical stimulation of tissue along the perimeter of the wound site. For example, hydrogels can be designed to include ionic conduction properties, and may be capable of generating large strains.

The systems, apparatuses, and methods described herein may provide significant advantages, many of which have already been discussed. Generally, the tape embodiments may promote an acceleration of wound closure through a variety of features and mechanisms. For example, when applied in the context of negative-pressure therapy, the tape may encourage epithelialization while the negative-pressure wound therapy promotes granulation, rather than having these processes separated into two discrete wound closure therapy steps.

The tape may also provide significant benefits to areas surrounding the wound that may typically be in contact with wound dressing covers. For example, the tape may help manage adherence of a wound cover, such as a drape, along the perimeter of the wound. The tape may both improve drape sealing to the wound perimeter to improve the application of negative-pressure wound therapy, as well as help minimize or avoid cellular trauma during dressing changes, particularly in the context of negative-pressure wound therapy. Additionally, the tape, when properly positioned, has the additional benefit of preventing drape shear along the wound epithelial edges to avoid damaging delicate cells on the surface of the healing wound edges. Furthermore, the tape may be constructed such that drape shear forces, which may result from multiple factors, do not transfer to the skin surface. For example, such forces may result from stretching and wrinkling due to the application of negative pressure to the wound dressing, or may be due to externally-applied shear forces during patient movement. The tape may also minimize or eliminate epithelial cell damage along the wound edge during drape and tissue interface removal and changes. The tape may also improve sealing, which may be beneficial for negative-pressure therapy, as well as optimize moisture management along the wound edge, which may help provide a preferred cellular environment for epithelialization.

Additionally, the tape embodiments disclosed herein may have benefits for users or caregivers. For example, some embodiments of the tape may be quickly applied with one hand, which may be less messy for a user than the use of a lotion product. The tape may also be beneficial for use as a sealing material in conjunction with negative-pressure wound therapy placements with wounds that are in body contours or other challenging locations which may be prone to leaks. In some embodiments, the tape may also provide training benefits for caregivers, as the tape may provide guidance for appropriate sizing of wound dressing components, such as a tissue interface or foam. For example, the tape may guide users to gradually reduce the size of a foam with each dressing change, to help counteract the clinically-improper tendency to overfill wounds with foam or other form of tissue interface.

While shown in a few illustrative embodiments, a person having ordinary skill in the art will recognize that the systems, apparatuses, and methods described herein are susceptible to various changes and modifications. Moreover, descriptions of various alternatives using terms such as “or” do not require mutual exclusivity unless clearly required by the context, and the indefinite articles “a” or “an” do not limit the subject to a single instance unless clearly required by the context. Components may be also be combined or eliminated in various configurations for purposes of sale, manufacture, assembly, or use. For example, in some configurations the dressing 102, the container 112, or both may be eliminated or separated from other components for manufacture or sale. In other example configurations, the controller 110 may also be manufactured, configured, assembled, or sold independently of other components.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described herein may also be combined or replaced by alternative features serving the same, equivalent, or similar purpose without departing from the scope of the invention defined by the appended claims.

What is claimed is:

1. A system for treating a tissue site, comprising:
a tape comprising a flexible material adapted to be placed in contact with a perimeter of the tissue site and including a non-adherent upper surface and a tissue-facing surface;
a tissue interface comprising a granulating surface;
a sealing member that is transparent and having an adherent material for coupling to an epidermis and operable to form a fluid seal; and
a negative-pressure subsystem for providing negative pressure to the tissue interface;
wherein the sealing member is adapted to not substantially adhere to the non-adherent upper surface of the tape.

2. The system of claim 1, wherein the tape is adapted to prevent shear forces from contacting the perimeter of the tissue site.

3. The system of claim 1, wherein the flexible material of the tape comprises a hydrogel.

4. The system of claim 1, wherein the tape is adapted to maintain an amount of moisture on the perimeter of the tissue site for optimizing epithelialization.

5. The system of claim 1, further comprising a reinforcement material placed in contact with the flexible material and adapted to provide structural support to the flexible material.

6. The system of claim 5, wherein the reinforcement material is positioned on a surface of the flexible material.

7. The system of claim 5, wherein the reinforcement material is substantially contained within the flexible material.

8. The system of claim 5, wherein the reinforcement material comprises a textile scrim.

9. The system of claim 1, wherein the tape further comprises a separating layer applied to a surface of the flexible material.

10. The system of claim 9, wherein the separating layer comprises a silicone layer.

11. The system of claim 9, wherein the separating layer is adapted to allow the tape to be formed into a roll.

12. The system of claim 9, wherein the separating layer is adapted to reduce attachment of an adhesive drape to the tape.

13. The system of claim 1, wherein the tape is formed with adequate strength to resist collapse under a compressive force transmitted by the sealing member when subjected to negative pressure.

14. A tape for use in a negative-pressure treatment system, comprising:
a flexible material adapted to be placed in contact with a perimeter of a wound; and
a reinforcement material placed in contact with the flexible material and adapted to provide structural support to the flexible material.

15. The tape of claim 14, wherein the flexible material comprises a hydrogel.

16. The tape of claim 14, further comprising a separating layer applied to a surface of the flexible material.

17. The tape of claim 16, wherein the separating layer comprises a silicone layer and is adapted to allow the tape to be formed into a roll.

18. The tape of claim 15, wherein the hydrogel is a porous, cationic hydrogel.

19. The tape of claim 14, wherein the flexible material comprises one or more of the following: cyanoacrylate sealant, fibrin sealant, silicone sealant, collagen sealant, hydrocolloid sealant, albumin sealant, hot-melt-type surgical paste, polymer gels such as polyurethanes, polyolefins, acrylics, and cellulosic (carboxymethyl cellulose), and alginates.

20. The tape of claim 14, wherein the flexible material further comprises human dermis cells.

21. The tape of claim 14, further comprising a conductive material adapted to transmit electrical current across the tape.

22. The tape of claim 21, wherein the conductive material comprises at least one of a carbon-impregnated fiber, a conductive polymer thread, or a thin metallic wire.

23. The tape of claim 21, wherein the conductive material is dispersed along each edge of the tape.

24. The tape of claim 14, further comprising a galvanic cell across the tape.

25. The tape of claim 14, further comprising a silk mat configured to slowly release epidermal growth factor.

26. The tape of claim 14, further comprising a material adapted to change colors according to temperature changes at the wound.

27. The tape of claim 15, wherein the hydrogel is configured to damage microbe membranes through electrostatic interactions.

28. The tape of claim 15, wherein the hydrogel is configured to provide mechanical stimulation to a perimeter of the wound.

29. The tape of claim 28, wherein the hydrogel further comprises ionic conductor properties.

30. The tape of claim 14, wherein the reinforcement material comprises a polyurethane elastomer.

31. The tape of claim 14, wherein the reinforcement material comprises a non-woven polyurethane elastomer.

32. A tape dispenser, comprising:
a tape spool;
a tape adapted to be contained in a roll by the tape spool, wherein the tape comprises:
a flexible material for disposing adjacent to a wound edge, and
a reinforcement material for providing structural support to the tape; and
a handle operatively fixed to the tape spool and configured for allowing a user to apply the tape around a perimeter of a wound.

33. The tape dispenser of claim 32, wherein the flexible material of the tape comprises a hydrogel.

34. The tape dispenser of claim 32, wherein the tape further comprises a separating layer having a silicone material and adapted to allow the tape to be formed into the roll.

35. A method of manufacturing a tape, comprising:
forming a flexible material for disposing adjacent to a wound edge;
forming a reinforcement material for providing structural support to the tape; and
disposing the reinforcement material on a first surface of the flexible material.

36. The method of claim 35, further comprising:
forming a separating layer adapted to reduce attachment of an adhesive drape to the tape; and
disposing the separating layer on a second surface of the flexible material.

37. The method of claim 35, wherein the flexible material comprises a hydrogel.

38. The method of claim 35, wherein the reinforcement material comprises a textile scrim.

39. A method of treating a wound site, comprising:
disposing a tape adjacent to a wound edge;
forming a fluid seal over the wound site and the tape; and
fluidly coupling a negative-pressure source to the wound site;
wherein the tape comprises:
a flexible material for disposing adjacent to a wound edge; and
a reinforcement material for providing structural support to the tape.

40. The method of claim 39, further comprising disposing a manifold adjacent to the wound site prior to forming a fluid seal over the wound site and the tape.

41. The systems, apparatuses, and methods substantially as described herein.
